(19) United States
(12) Patent Application Publication
(10) Pub. No.: US 2006/0275339 A1
(43) Pub. Date: Dec. 7, 2006

(54) Use of antiseptic active principles in pmma bone cements

(75) Inventors: Frank Schilke, Weiterstadt (DE); Berthold Nies, Fraenkisch-Crumbach (DE); Hanns Pietsch, Hamburg (DE)

Correspondence Address: THE WEBB LAW FIRM, P.C., 700 KOPPERS BUILDING, 436 SEVENTH AVENUE, PITTSBURGH, PA, 15219, US

(73) Assignee: Biomet Deutschland, Berlin (DE)

(21) Appl. No.: 10/564,372

(22) PCT Filed: Jul. 16, 2004
(86) PCT No.: PCT/DE04/01571

§ 371 Date: Jun. 8, 2006

(30) Foreign Application Priority Data
Jul. 18, 2003 (DE) 10332680.4

(51) Int. Cl. A61K 31/155; A61K 9/14

(52) U.S. Cl. 424/426; 424/486; 514/635

(57) ABSTRACT

The invention concerns the use of polyhexamethylene biguanide in PMMA bone cements, which preferably do not contain antibiotics. The cements exhibit a concentration of active principle not exceeding 1 wt. % relative to the total weight of the cement, which is a concentration sufficiently high to prevent microbial colonization of the cement surface. The cements do not cause durable alteration of the healing process.

The invention relates to the use of antiseptic active principles in polymethyl methacrylate bone cements (PMMA cement) with an active-principle concentration which is sufficient to prevent microbial colonisation of the cement surface.

Conventional medicament-containing bone cements consist of a PMMA or PMMA copolymer powder in which, inter alia, the pulverulent medicament is distributed. After admixing of a monomer liquid (with an activator), polymerisation occurs. The cured bone cement is then a polymer mass from which the medicament located in the surface layer is released.

In order to prevent septic inflammation reactions after microbial colonisation of the cement and/or the adjacent tissue, antibiotics are used as medicament in conventional bone cement. However, the widespread use of antibiotics in bone cements is increasingly resulting in the development of antibiotic-resistant bacterial strains, meaning that it is no longer possible under certain circumstances completely to prevent wound infections. The use of more recent antibiotics is likewise not a long-term solution since bacterial strains which are resistant to the new medicament will form in the foreseeable future.

EP 701 824 (Merck Patent GmbH) describes a process for the production of active-principle-containing bone cements which may also comprise, inter alia, antibiotics or antiseptics.

WO 98/07456 (Merck Patent GmbH) relates to a process for the production of active-principle-containing bone cements and bone replacement materials or implantable pharmaceutical depots produced therefrom which may also comprise, inter alia, antibiotics or antiseptics.

EP 202 445 (Merck Patent GmbH) relates to a pharmaceutical depot which can be implanted in the body for controlled delayed release of cytostatics that, in addition to a cytostatic, may also comprise an antibiotic and/or antiseptic.

EP 234 004 (Merck Patent GmbH) describes an implantable pharmaceutical depot which comprises antibiotics and antiseptics for increasing or augmenting the action of the chemotherapeutic agent.

The object of the invention is to replace the antibiotic in conventional bone cements by a novel medicament without adversely affecting the antibacterial action on the surface of the cement. The novel medicament should, owing to its different mechanism of action, prevent the formation of resistant bacteria in the long term. The novel medicament should be selected in nature and concentration in such a way that the antibacterial action is ensured, but wound healing is not significantly impaired.

The object is achieved by the use of antiseptic active principles in a PMMA bone cement with an active-principle concentration which is sufficient to prevent microbial colonisation of the cement surface. The PMMA bone cement preferably comprises no antibiotic.

Suitable antiseptics are compounds from the following groups:
quaternary ammonium compounds, such as hexadecyidimethylethylammonium ethosulfate or didecyldimethylammonium chloride,
amine oxides, such as N-alkyl(C10-C18)-N,N-dimethylamine N-oxide or N-alkyl(C10-C18)-N,N-diethylamine N-oxide,
pyridine derivatives, such as octenidine dihydrochloride,
guanidines, such as polyhexamethylenebiguanide hydrochloride, and/or
10-undecylenic acid amides, such as 10-undecylenic acid N-ethanolamide.

Preference is given to the addition of polyhexamethylenebiguanide in a maximum amount of 1% by weight, based on the total weight of the cement. Still more preference is given to a maximum amount of 0.5% by weight, with much more preference being given to an amount of from 0.025 to 0.5% by weight. A maximum amount of 0.155% by weight of polyhexamethylenebiguanide is most preferred. In accordance with the invention, it is also possible to add more than one antiseptic active principle.

[image]

As can be seen from FIGS. 1a and 1b, admixing of only 0.155% by weight of polyhexamethylenebiguanide with a PMMA bone cement (PALAMED® plain) has the same (or higher) biological efficacy in preventing colonisation of the cement surface with germs as the admixing of 0.86% by weight of gentamicin (antibiotic) used for comparison.

The production of the bone cement according to the invention is described in greater detail with reference to two examples.

### EXAMPLE 1

97.3 mg of polyhexamethylenebiguanide hydrochloride were mixed into 18.8 g of Palamed liquid (consisting of methyl methacrylate, N,N-dimethyl-p-toluidine and dye). The homogeneous solution was mixed with 44 g of Palamed powder (plain; without gentamicin) in a vacuum mixing system in accordance with the manufacturer's instructions. The mixture was introduced into moulds and cured.

### EXAMPLE 2

5.3 g of zirconium dioxide were mixed with a solution of 97.3 mg of polyhexamethylenebiguanide hydrochloride in 400 mg of water. The water was removed by freeze-drying. The antiseptic-containing zirconium dioxide was subsequently mixed with 38.3 g of poly(methyl methacrylate-co-methyl acrylate) and 0.44 g of dibenzoyl peroxide. The resultant powder was added to a solution of 0.4 g of N,N-dimethyl-p-toluidine in 18.4 g of methyl methacrylate, and the two were mixed intensively. The mixture was introduced into moulds and cured.

1-6. (canceled)

7. A method of preventing microbial colonization of a polymethyl methacrylate (PMMA) bone cement surface, comprising the step of admixing polyhexamethylene biguanide with a PMMA bone cement, wherein the active principle concentration of the polyhexamethylene biguanide is 1% or less by weight of the total amount of the PMMA bone cement.

8. The method of claim 7, wherein the PMMA bone cement admixture does not contain an antibiotic.

9. The method of claim 7, wherein the PMMA bone cement admixture does not adversely affect the wound-healing process in the long term and does not significantly impair the curing process of the bone cement.

10. The method of claim 7, wherein the active principle concentration of the polyhexamethylene biguanide is in an amount of from 0.025 to 0.5% by weight of the total amount of the PMMA bone cement.

11. The method of claim 10, wherein the active principal concentration of the polyhexamethylene biguanide is in a maximum amount of 0.155% by weight of the total amount of the cement.

12. A medical implant produced from bone cements according to claim 7.

13. The method of claim 8, wherein the PMMA bone cement admixture does not adversely affect the wound-healing process in the long term and does not significantly impair the curing process of the bone cement.

14. The method of claim 8, wherein the active principle concentration of the polyhexamethylene biguanide is in an amount of from 0.025 to 0.5% by weight of the total amount of the PMMA bone cement.

15. The method of claim 9, wherein the active principle concentration of the polyhexamethylene biguanide is in an amount of from 0.025 to 0.5% by weight of the total amount of the PMMA bone cement.

16. The method of claim 7, wherein the polyhexamethylene biguanide is an antiseptic.
